(19) United States
(12) Patent Application Publication
(10) Pub. No.: US 2007/0003643 A1
(43) Pub. Date: Jan. 4, 2007

(54) Soybean Jam

(75) Inventor: Ritsuko Sakai, Yokohama-Shi, Kanagawa (JP)

Correspondence Address: NORRIS, MCLAUGHLIN & MARCUS, P.A., 875 THIRD AVE, 18TH FLOOR, NEW YORK, NY, 10022, US

(73) Assignee: Yugenkaisha Sakai, Yokohama-shi (JP)

(21) Appl. No.: 11/428,685

(22) Filed: Jul. 5, 2006

Related U.S. Application Data
(63) Continuation-in-part of application No. 10/984,063, filed on Nov. 9, 2004, now abandoned.
Application No. 10/984,063 is a division of application No. 10/155,091, filed on May 24, 2002, now Pat. No. 6,833,152.

(30) Foreign Application Priority Data
Oct. 31, 2001 (JP) 2001-344290

(51) Int. Cl. A61K 36/48

(52) U.S. Cl. 424/757

(57) ABSTRACT

The present invention provides a soybean jam that can increase the opportunities for consuming soybean, which is known as a health food, particularly in a Western style diet with few chances to eat them. The present invention also provides a method for manufacturing the soybean jam comprising the steps of manufacturing a soybean paste by grinding and kneading soybean grains after boiling, and forming a jam by mixing the soybean paste with other ingredients of the jam including pectin, sugar and organic acids followed by boiling down the mixture. The soybean jam obtains a large proportion of the soybean component.

This application is a continuation-in-part of U.S. Ser. No. 10/984,063, filed Nov. 9, 2004 which is a divisional of U.S. Ser. No. 10/155,091, filed May 24, 2002, now U.S. Pat. No. 6,833,152.

### BACKGROUND OF THE INVENTION

1. Field of the Invention

The present invention relates to a method for improving the opportunities for eating soybean by providing soybean, which is known as a health food, in a novel edible form, such as a jam, thereby enabling an increase in the consumption of soybean, even in a Western style diet, such as the United States, where current soybean intake is relatively low.

2. Description of the Related Art

Soybean has become known as a health food in recent years. While soybean is known as an excellent protein source abundant in edible fibers, oligosaccharides, potassium, calcium, magnesium, phosphor and iron, the opportunities for eating soybean have decreased with the change of lifestyle in Japan. Although processed soybean products such as fermented soybeans (natto), soybean curd (tofu) and soybean paste (miso) have been used as foodstuffs suitable for Japanese style diet and have become known abroad, especially in the United States, as health foods, they are generally not a familiar part of a Western style diet, and there are few opportunities to eat soybean. When soybean is supplied in a novel food form, it will be expected that more soybean is consumed in the Japanese style diet as well as in the Western style diet, in addition to consuming as the foodstuffs in traditional forms, such as fermented soybeans and soybean curd.

JP 09-56356 discloses adding a yuzu citron wafer or lemon wafer, but only for the purpose of affecting the flavor. While such additives do indeed have some pectin, the total amount of pectin added to the product would be nominal, since the purpose of the addition is only for flavor. Such a small amount of pectin cannot make soybean paste jam-like. JP 09-56356 SOYBEAN JAM is not a jam-like pasty healthy food with pectin which makes a jam-like soybean paste, but is essentially “anko” which has stickiness derived from the structure of soybean protein thermally coagulated around soybean starch granule. “An” or “anko” is a part of an old Japanese-style diet in which adzuki beans are boiled, ground, salted, and sugar added, and then cooked and kneaded to obtain the final “an or anko”. Examples which contain “an or anko” are Japanese sweets such as “shiruko”, which is a sweet red-bean soup with rice cake. No fruits are used in the production of “an or anko”, and thus there are differences in taste and flavor between “jam” and “an or anko”. The lack of pectin in “an or anko” indicates no sourness or stickiness. One exception is “an or anko” which contains a small amount of “yuzu” or citron for fragrance, as in JP 09-56356, but there is no sourness or stickiness from the addition of pectin. In addition, the producers and the shops for “an or anko” are different from those that produce “jam”.

JP 05-304922, JP 58-152459, JP 61-9259, JP 61-119161 and JP 63-126461 disclose essentially “anko” including no pectin.

### SUMMARY OF THE INVENTION

Accordingly, it is an object of the present invention is to increase the opportunities to eat soybean, and to provide soybean foods suitable for the Western style diet, such as the United States, where current soybean consumption is low.

The present invention for attaining the forgoing objects provides a soybean jam using a soybean paste prepared by grinding and kneading to a paste after boiling soybean grains, wherein this soybean paste is mixed with other ingredients of the jam including pectin, sugar and organic acids, followed by boiling down the mixture.

Another object of the present invention is to provide a method for manufacturing a soybean jam comprising the steps of manufacturing a soybean paste by grinding and kneading soybean grains after boiling, and forming a jam by mixing the soybean paste with other ingredients of the jam including pectin, sugar and organic acids, followed by boiling down the mixture.

Granular pieces of the soybean grains prepared by crushing the soybean grains may be added to the soybean paste.

Preferably, granular pieces of crushed grape or raisin are added to the soybean paste.

A flavoring material such as a liquor or lemon jounce may be added to the soybean paste.

It is desirable to remove the epidermis of boiled soybean in advance of crushing the soybean grains after boiling.

Raisin, grape, strawberry or orange may be used as an ingredient of the jam.

Preferably, sugar is supplemented with raw sugar, honey or syrup when a fruit deficient in sugar is used as the ingredient of the jam.

Preferably, organic acid components are supplemented with a sour fruit juice such as a lemon juice when a fruit deficient in organic acids is used as the ingredient of the jam.

Soybean as used in the present invention includes green soybean, namely immature soybean, in addition to mature soybean. Accordingly, the soybean jam and soybean paste may be manufactured using green soybean grains in place of the mature soybean grains.

### BRIEF DESCRIPTION OF THE DRAWINGS

FIG. 1 illustrates an example of the method for manufacturing the soybean jam according to the present invention.

### DESCRIPTION OF THE PREFERRED EMBODIMENTS

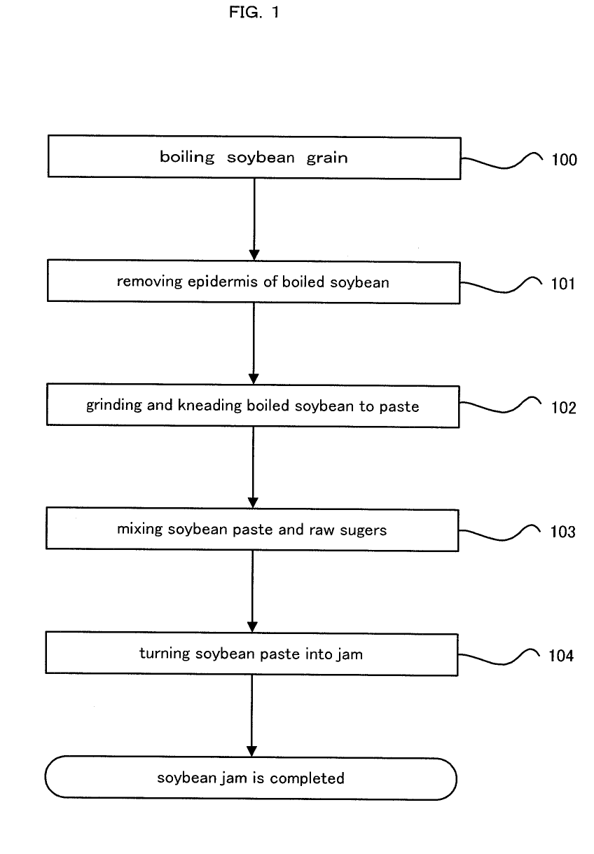

An example of the method for manufacturing the soybean jam according to the present invention, and an example of the method for manufacturing the soybean paste used as an ingredient of the soybean jam will be described in detail with reference to FIG. 1.

The soybean jam can be manufactured by mixing a soybean paste and an ingredient of the jam containing pectin, sugars and organic acids and by boiling down the mixture.

The soybean paste can be manufactured by grinding soybean after boiling the soybean grains, followed by thoroughly kneading to form a paste. The epidermis of boiled soybean is preferably removed before grinding.

At least the three components pectin, sugars and organic acids should be contained in a given proportion as ingredients of the jam, because the jam is only formed by an interaction among pectin, sugars and organic acids.

Accordingly, it is possible to use raw materials containing the three components of pectin, sugars and organic acids, such as raisin, grape and orange, as the ingredients of the soybean jam according to the present invention.

A standard proportion among pectin, sugars and organic acids necessary for forming the jam containing the ingredient as described above is 1.0 to 1.5% by weight of pectin, 65 to 70% by weight of sugars and 0.4 to 0.45% by weight of organic acids, relative to the total weight of all ingredients of the jam. Any starting material containing pectin, sugars and organic acids in this proportion can be formed into the jam by boiling.

Since a fruit that is used as an ingredient of the jam contains all of the three components (pectin, sugars and organic acids), the soybean jam can be basically manufactured using only the fruit and soybean paste. However, when a fruit relatively deficient in sugars as compared with the standard proportion of pectin, sugars and organic acids as described above is used, sugars may be supplemented with raw sugar, honey or syrup. When the fruit is deficient in organic acids, they may be supplemented with a sour fruit juice such as a lemon juice. Lemon also serves for adding flavor in the soybean jam. Liquors may be used for adding flavor to the soybean jam.

The method for manufacturing the soybean jam will be described hereinafter with reference to FIG. 1.

The soybean grains are boiled at first (step 100). Then, the epidermis of boiled soybean is removed (step 101). Subsequently, boiled soybean is ground and thoroughly kneaded to a paste, preferably with a pestle and mortar, thereby obtaining a paste of soybean required for manufacturing the soybean jam (step 102).

After obtaining the soybean paste by the steps 100 to 102, raw sugars or honey is mixed with the soybean paste as a sugar component. The proportion of mixing of the soybean paste and sugars such as raw sugar is 1:about 0.2. A liquor or lemon juice may be added as a flavoring material into the soybean paste (step 103).

A syrup and fruit juice is further added in the soybean paste together with sugars such as raw sugars, and the mixture is boiled. Then, the soybean paste turns into a jam by an interaction among the pectin component and organic acids in the fruit, sugars in the fruit, sugars in raw sugar, and honey and syrup in the soybean paste, thereby completing the soybean jam (step 104).

The fruit to be added to the soybean paste in the step 104 may comprise, for example, finely crushed pieces of raisin or grape.

While all the boiled soybean is made to be a paste in the step 102, a part of the boiled soybean may be crushed as granules to mix with the soybean paste.

The soybean jam manufactured as described above may be directly eaten. However, it is usually eaten by spreading on a slice of bread as a strawberry jam is. The soybean paste used for manufacturing the soybean jam may be also used for base materials of various dishes, and use of the soybean paste is not restricted to manufacture of the soybean jam. Further, the soybean jam may be added to peanut paste, which is common and widely consumed in countries outside of Japan, especially in the United States.

While manufacturing of the soybean paste and soybean jam using matured soybean grains has been described in the example, the soybean paste and soybean jam may be manufactured by the same manufacturing method as described above using green, immature soybean in place of matured soybean.

A jam containing a large proportion of the soybean component can be obtained in the present invention. Since it is made possible to eat soybean in a novel form such as soybean jam, the ability to eat soybean in the daily diet increases by making soybean to be a more familiar health food. Since the jam is eaten together with bread, more soybean may be consumed in a Western style diet, where presently soybean is seldom eaten.

### EXAMPLE 1

Soybean paste 233 gr., honey 4 gr., black raw sugar 24 gr., Sensoto sugar 46 gr., pectin powder 3 gr., soybean nutriment solved and suspended water 75 ml. and orange 58 gr. are mixed and boiled down into a soybean-based jam-like pasty healthy food, with delicious orange taste and health by soybean nutriment, as steps 103 and 104 in FIG. 1. The weight ratio is calculated to the soybean paste 100, the sugars (black raw sugar, Sensoto sugar and honey) being 32 parts, the pectin powder 1.3 parts and the orange 25 parts.

Above Sensoto sugar is a raw crystallized sugar manufactured in the process of drawing from sugarcane, boiling down and twice washing through centrifugal process. Sensoto sugar has rich nutriment such as mineral and organism contained in sugarcane.

Pectin powder is a pure pectin produced from natural fruits or vegetables.

### EXAMPLE 2

Soybean paste 233 gr., honey 4 gr., black raw sugar 24 gr., Sensoto sugar 46 gr., pectin powder 3 gr., soybean nutriment solved and suspended water 75 ml. and orange peel 58 gr. are mixed and boiled down into a soybean-based jam-like pasty healthy food, with delicious and sweet orange taste and health by soybean nutriment as steps 103 and 104 in FIG. 1. The weight ratio is calculated to the soybean paste 100, the sugars (black raw sugar, Sensoto sugar and honey) 32, the pectin powder 1.3 and the orange peel 25.

### EXAMPLE 3

Soybean paste 233 gr., honey 4 gr., black raw sugar 24 gr., Sensoto sugar 46 gr., pectin powder 3 gr., soybean nutriment solved and suspended water 75 ml. and strawberry 65 gr. are mixed and boiled down into a soybean-based jam-like pasty healthy food, with delicious strawberry taste and health by soybean nutriment as steps 103 and 104 in FIG. 1. The weight ratio is calculated to the soybean paste 100, the sugars (black raw sugar, Sensoto sugar and honey) 32, the pectin powder 1.3 and the strawberry 28.

### EXAMPLE 4

Soybean paste 233 gr., honey 4 gr., black raw sugar 24 gr., Sensoto sugar 46 gr., pectin powder 3 gr., soybean nutriment solved and suspended water 75 ml. and blueberry 65 gr. are mixed and boiled down into a soybean-based jam-like pasty healthy food, with delicious blueberry taste and health by soybean nutriment as steps 103 and 104 in FIG. 1. The weight ratio is calculated to the soybean paste 100, the sugars (black raw sugar, Sensoto sugar and honey) 32, the pectin powder 1.3 and the blueberry 28.

### EXAMPLE 5

Soybean paste 233 gr., honey 4 gr., black raw sugar 24 gr., Sensoto sugar 46 gr., pectin powder 2.5 gr., soybean nutriment solved and suspended water 75 ml. and apple 58 gr. are mixed and boiled down into a soybean-based jam-like pasty healthy food, with delicious apple tast and health by soybean nutriment as steps 103 and 104 in FIG. 1. The weight ratio is calculated to the soybean paste 100, the sugars (black raw sugar, Sensoto sugar and honey) 32, the pectin powder 1.0 and the apple 25.

### EXAMPLE 6

Soybean paste 233 gr., honey 4 gr., black raw sugar 24 gr., Sensoto sugar 46 gr., pectin powder 3 gr., soybean nutriment solved and suspended water 75 ml. and banana 58 gr. are mixed and boiled down into a soybean-based jam-like pasty healthy food, with delicious banana tast and health by soybean nutriment as steps 103 and 104 in FIG. 1. The weight ratio is calculated to the soybean paste 100, the sugars (black raw sugar, Sensoto sugar and honey) 32, the pectin powder 1.3 and the banana 25.

### EXAMPLE 7

Soybean paste 233 gr., honey 4 gr., black raw sugar 24 gr., Sensoto sugar 46 gr., pectin powder 3 gr., soybean nutriment solved and suspended water 75 ml. and peanut butter 58 gr. are mixed and boiled down into a soybean-based jam-like pasty healthy food, with delicious peanut butter tast and health by soybean nutriment as steps 103 and 104 in FIG. 1. The weight ratio is calculated to the soybean paste 100, the sugars (black raw sugar, Sensoto sugar and honey) 32, the pectin powder 1.3 and the peanut butter 25.

### EXAMPLE 8

Soybean paste 233 gr., honey 4 gr., black raw sugar 24 gr., Sensoto sugar 46 gr., pectin powder 2 gr., soybean nutriment solved and suspended water 75 ml. and chocolate syrup 58 gr. are mixed and boiled down into a soybean-based jam-like pasty healthy food, with delicious chocolate tast and health by soybean nutriment as steps 103 and 104 in FIG. 1. The weight ratio is calculated to the soybean paste 100, the sugars (black raw sugar, Sensoto sugar and honey) 32, the pectin powder 0.9 and the chocolate syrup 25.

### EXAMPLE 9

Soybean paste 233 gr., honey 4 gr., black raw sugar 24 gr., Sensoto sugar 46 gr., pectin powder 4.5 gr., soybean nutriment solved and suspended water 75 ml. and strawberry 65 gr. are mixed and boiled down into a soybean-based jam-like pasty healthy food, with delicious strawberry taste and health by soybean nutriment as steps 103 and 104 in FIG. 1. The weight ratio is calculated to the soybean paste 100, the sugars (black raw sugar, Sensoto sugar and honey) 32, the pectin powder 1.9 and the strawberry 28.

This soybean-based jam-like pasty healthy food is rather expensive because of rich pectin. More rich pectin would make it lose market value on higher cost.

### COMPARATIVE EXAMPLE 1

Soybean paste 233 gr., honey 4 gr., black raw sugar 24 gr., Sensoto sugar 46 gr., pectin powder 1.5 gr., soybean nutriment solved and suspended water 75 ml. and orange 58 gr. are mixed and boiled down into a soybean-based healthy food. This healthy food lacks paste temper and is not good to eat for a person who usually takes butter and jam with bread and cannot take pure soybean paste.

The weight ratio is calculated to the soybean paste 100, the sugars (black raw sugar, Sensoto sugar and honey) 32, the pectin powder 0.6 and the orange 25.

### COMPARATIVE EXAMPLE 2

Soybean paste 233 gr., honey 4 gr., black raw sugar 24 gr., Sensoto sugar 46 gr., pectin powder 1.3 gr., soybean nutriment solved and suspended water 75 ml. and apple 58 gr. are mixed and boiled down into a soybean-based healthy food. This healthy food lacks paste temper and is not good to eat for a person who usually takes butter and jam with bread and cannot take pure soybean paste.

The weight ratio is calculated to the soybean paste 100, the sugars (black raw sugar, Sensoto sugar and honey) 32, the pectin powder 0.6 and the apple 25.

The delicious food material in the present invention is not restricted in weight ratio to soybean paste. The ratio should be properly decided meeting the taste of the consumers, wherein the ratio of material does not excess beyond that of healthy food.

The sugars used in the present invention are not restricted to black raw sugar and Sensoto sugar, but any other sugars may be selected meeting the taste of the consumers, wherein the ratio of sugars does not excess beyond that of healthy food. The ratio of sugars is less than 70, preferably less than 60 to soybean paste (100).

The fruit or berry used in the present invention is not restricted to orange, orange peal, apple, and banana, but any other fruit or berry may be selected meeting the taste of the consumers, wherein the ratio of material does not excess beyond that of healthy food. The ratio of fruit or berry is less than 50, preferably less than 40 to soybean paste (100).

The nut used in the present invention is not restricted to peanut butter and chocolate syrup, but any other nut may be selected meeting the taste of the consumers, wherein the ratio of material does not exceed beyond that of healthy food. The ratio of nut is less than 50, preferably less than 40 to soybean paste (100).

Using only the pectin included in fruit and berries is insufficient to make soybean paste jam-like pasty. If only natural pectin in fruit and berries would be applied for making soybean paste jam-like pasty, an excessive amount of fruit and berry would need to be used. It might be sweet and delicious, but it would become like conventional jam lacking in healthy nutriment of soybean.

Additional pectin of 0.8-1.9 weight ratio to the soybean paste (100) in the present invention, the inventor found, is very effective to make soybean paste jam-like pasty, keeping both of enough nutriment of soybean and delicious jam-like paste, good to eat for a person who usually takes butter and jam with bread and cannot take pure soybean paste.

What is claimed is:

1. A soybean-based jam-like pasty healthy food manufactured by boiling down of soybean paste, pectin and food material mixture, with weight ratio as 100 parts of the soybean paste, 0.8-1.9 parts of the pectin, and an appropriate quantity of the food material.

2. The soybean-based jam-like pasty healthy food according to claim 1, wherein the food material comprises fruit and/or berry, and sugar.

3. The soybean-based jam-like pasty healthy food according to claim 1, wherein the food material comprises nuts and sugar.

4. The soybean-based jam-like pasty healthy food according to claim 1, wherein the food material comprises one or more of fruit, berry and nuts, along with sugar, and wherein the fruit, berry and/or nuts is crushed into a paste.

5. The soybean-based jam-like pasty healthy food according to claim 1, wherein the food material comprises granular pieces of fruit or berry.

6. The soybean-based jam-like pasty healthy food according to claim 1, wherein further soybean nutriment solved and suspended water, which is attained in a boiling process of raw soybean for the soybean paste, is added in the boiling-down mixture.

7. The soybean-based jam-like pasty healthy food according to claim 1, wherein further granular pieces of soybean grains are added in the boiling-down mixture.

8. The soybean-based jam-like pasty healthy food according to claim 1, wherein the soybean paste has no soybean epidermis.

9. The soybean-based jam-like pasty healthy food according to claim 1, wherein the soybean paste is made from immature green soybean.

10. The soybean-based jam-like pasty healthy food according to claim 1, wherein the food material comprises sugars and a food material selected from orange, orange peel, apple, banana, peanut butter and chocolate syrup, with weight ratio of the soybean paste 100, the pectin 1.5, the sugars 32 and the food material 25.

11. The soybean-based jam-like pasty healthy food according to claim 1, wherein the food material comprises sugars and a food material selected from strawberry and blueberry, with weight ratio of the soybean paste 100, the pectin 1.5, the sugars 32 and the food material 28.

12. A soybean-based jam-like pasty healthy food comprising soybean paste, pectin and food material mixture, with weight ratio as 100 parts of the soybean paste, 0.8-1.9 parts of the pectin, and an appropriate quantity of the food material.

13. The soybean-based jam-like pasty healthy food according to claim 12, wherein the food material comprises fruit and/or berry, and sugar.

14. The soybean-based jam-like pasty healthy food according to claim 12, wherein the food material comprises nuts and sugar.

15. The soybean-based jam-like pasty healthy food according to claim 12, wherein the food material comprises sugar and a paste of one or more of fruit, berry and nuts.

16. The soybean-based jam-like pasty healthy food according to claim 12, wherein the food material comprises granular pieces of fruit or berry.

17. The soybean-based jam-like pasty healthy food according to claim 12, further comprising soybean nutriment solved and suspended water obtained in a boiling process of raw soybean for the soybean paste.

18. The soybean-based jam-like pasty healthy food according to claim 12, further comprising granular pieces of soybean grains.

19. The soybean-based jam-like pasty healthy food according to claim 12, wherein the soybean paste has no soybean epidermis.

20. The soybean-based jam-like pasty healthy food according to claim 12, wherein the soybean paste is made from immature green soybean.

21. The soybean-based jam-like pasty healthy food according to claim 12, wherein the food material comprises sugars and a food material selected from orange, orange peel, apple, banana, peanut butter and chocolate syrup, with weight ratio of the soybean paste 100, the pectin 1.5, the sugars 32 and the food material 25.

22. The soybean-based jam-like pasty healthy food according to claim 12, wherein the food material comprises sugars and a food material selected from strawberry and blueberry, with weight ratio of the soybean paste 100, the pectin 1.5, the sugars 32 and the food material 28.
